(19) United States
(12) Patent Application Publication
(10) Pub. No.: US 2026/0311839 A1
(43) Pub. Date: Oct. 8, 2026

(54) Improved Post-Myocardial Infarct Exercise

(71) Applicant: BIOTRONIK SE & Co. KG, Berlin (DE)

(72) Inventors: Dominic WIST, Berlin (DE); Bjoern DIEM, Berlin (DE); Volker LANG, Berlin (DE)

(73) Assignee: BIOTRONIK SE & Co. KG, Berlin (DE)

(21) Appl. No.: 19/481,490

(22) PCT Filed: Apr. 26, 2024
(86) PCT No.: PCT/EP2024/061516

§ 371 Date: Nov. 4, 2025

(30) Foreign Application Priority Data
May 11, 2023 (EP) 23172837.9

(51) Int. Cl. G16H 20/30; G16H 10/60; G16H 40/67

(52) U.S. Cl. CPC G16H 20/30; G16H 10/60; G16H 40/67

(57) ABSTRACT

An apparatus for providing exercise assistance includes means for receiving dynamical patient data comprising at least one physiological parameter and means for receiving exercise data including a current exercise parameter. The apparatus further includes means for providing at least one new and/or updated exercise parameter of an ongoing and/or an upcoming patient exercise, based at least in part on the dynamical patient data.

### CROSS-REFERENCE TO RELATED APPLICATIONS

This application is the United States National Phase under 35 U.S.C. § 371 of PCT International Patent Application No. PCT/EP2024/061516, filed on Apr. 26, 2024, which claims the benefit of European Patent Application No. 23172837.9, filed on May 11, 2023, the disclosures of which are hereby incorporated by reference herein in their entireties.

### TECHNICAL FIELD

The present invention relates to an apparatus, a respective system and computer program for improved exercise assistance for patients with impaired health.

### BACKGROUND

Patients with impaired health, like post-myocardial infarct (MI) patients, commonly undergo rehabilitation measures including, e.g., an exercise plan to accelerate their recovery. For example, physiotherapists and other health experts may assist during exercise sessions for post-MI patients which are adapted to the specific needs of the patient. However, often, only a part of the lengthy recovery may be supervised by trained personnel and in their further recovery, patients rely, e.g., on standard training apps, which are, however, designed for healthy people. These are not adapted to the specific post-infarction needs and could even be dangerous in these patients.

There is therefore still a need to further improve solutions, including devices, systems, methods, and computer programs, for exercise assistance for patients with impaired health.

The above need is at least in part met by the various aspects described herein.

The present disclosure is directed toward overcoming one or more of the above-mentioned problems, though not necessarily limited to embodiments that do.

### SUMMARY

According to an aspect of the invention, an apparatus for providing exercise assistance is provided. The apparatus comprises means for receiving dynamical patient data comprising at least one physiological parameter and means for receiving exercise data comprising a current exercise parameter. The apparatus further comprises means for determining a new and/or updated exercise parameter of an ongoing and/or an upcoming patient exercise, based at least in part on the dynamical patient data.

The apparatus may provide improved suggestions for ongoing and/or upcoming exercise sessions, reduce the risk associated with badly adjusted exercise suggestions, especially to users with a poor health condition. This may thus improve success chances of the exercise-assisted recovery.

The apparatus may be a mobile device, a (remote) server, a cloud-based server, a wearable device, etc.

It is to be understood that herein the term ‘exercise’ generally refers to a physical exercise, such as a sports activity.

The means for receiving dynamical patient data and/or the means for receiving the exercise data may be any wireless data transmission/receiving components/interfaces, e.g., antennas, and/or be based on wires and/or any other electrical conductor. The according communication may, for example, be based on Bluetooth, radio broadcast, infrared, satellite, and/or microwave.

The dynamical patient data comprise at least one physiological parameter. The at least one physiological parameter may for example be provided by one of the following: an at least partly implantable device, a wearable device (e.g., a smart watch), an attached device (e.g., affixed to the skin or affixed as a piercing), and a mobile medical device. One or more devices may be involved in acquiring the dynamical patient data. The involved devices may acquire different physiological parameters each or the involved devices may cooperate to determine the same physiological parameter for improved accuracy.

The exercise data comprise at least one current exercise parameter. The at least one current exercise parameter may for example be provided in form of an exercise plan provided to the patient. The at least one current exercise parameter may relate to an exercise type, e.g., running, swimming, cycling, muscle-building training, stretching, etc. or to a more specific exercise, e.g., biceps curls, squats, stretching of a specific muscle, etc. It may further comprise a measure associated with the respective exercise, e.g., a number of sets and/or repetitions (e.g., three sets of twelve repetitions, each), a distance, calorie goal, and/or time limit (for running, cycling, swimming, etc.) and/or any combination of the exercise parameters described herein. The exercise data may be provided by a database. The patient might provide user input to the apparatus to inform the apparatus that they perform the exercise according to the respective at least one current exercise parameter.

The means for determining at least one new and/or updated exercise parameter of an ongoing and/or an upcoming patient exercise may, e.g., be based at least in part on the physiological data and/or exercise data. They may use any suitable (semi-)automated calculation mechanism for the prediction of suitable exercise parameter(s), e.g., in form of an exercise plan.

The input to the prediction may be patient specific (demographic data like age, gender, information on diseases, and/or data from prior exercise testing) and/or population based (guidelines, data sets from other patients). From these data the prediction algorithm may calculate boundary conditions for maximum heart rate, maximum load, optimal type of exercise and convert them into an exercise plan which may state: How many minutes on which workload on a home exercise bike, or a suggestion for power walking or jogging.

In an example, the dynamical patient data may be provided by an implantable device.

Implantable devices, which may also be partly implantable devices, are particularly suitable for continuous monitoring of one or more physiological parameters.

Possible implants may comprise, e.g., a cardiac sensor, an accelerometer, a neurostimulation device, an electrocardiography, ECG, device, etc. and/or any other device for acquiring physiological data. The implant may have further functions beyond acquiring physiological data, e.g., therapeutical functions. For example, a neurostimulation device may, e.g., provide (therapeutical) neurostimulation (e.g., for pain relief) and measure one or more physiological parameters (e.g., associated with pain perceived by the patient).

In an exemplary embodiment, the dynamical patient data received by the apparatus may comprise at least one of: a cardiovascular parameter, a respiratory parameter, and a motion-related parameter.

For example, the cardiovascular parameter may be, e.g., the blood pressure, heart rate, etc. of the patient. The respiratory parameter may, e.g., be the breath frequency and/or depth. The motion-related parameter may, e.g., be a number of steps per day.

These parameters may be particularly suitable to determine the updated and/or new exercise parameters based thereon.

In an example, the apparatus may further comprise means for receiving static patient data and/or external data and the means for determining may further be configured for determining the at least one new and/or updated exercise parameter based at least in part on the static patient data and/or the external data.

The static patient data may for example comprise height, weight, gender, medication of the patient, a health record of the patient comprising, e.g., previous medical conditions, etc. The apparatus may for example adjust the suggested exercise plan when the static patient data, e.g., their weight, indicates that a certain exercise may not be suitable. The apparatus may then for example suggest cycling rather than jogging. The apparatus may comprise means for accessing an electronic health record (EHR) and/or database storing, e.g., static patient data, external data, and/or a log of exercise data. The apparatus may, e.g., access the respective electronic patient file of the EHR/database via a patient identification (ID) or an implant ID. Generally, the apparatus may access any data described herein that may be stored in such electronic patient file and may alternatively or additionally store any data described herein in the respective electronic patient file. The EHR/database may be comprised in a remote server and/or a cloud based server, for example.

The external data may for example comprise the current weather or a weather forecast, opening hours of exercise facilities, etc. For example, the apparatus may suggest indoor activities when a maximum temperature is exceeded which might render running outside harmful rather than beneficial.

In an example, the apparatus may further comprise means for determining a new and/or updated exercise plan for an ongoing and/or an upcoming patient exercise, based at least in part on the at least one new and/or updated exercise parameter.

This may increase user satisfaction and recovery success chances.

The exercise plan may comprise a list of exercises with further exercise-related details for each exercise for a list of exercise sessions. Each session might be assigned a suggested time window for when to schedule the respective exercise session. The suggestions for upcoming exercise sessions might be preliminary such that they might be adjusted based at least in part on data acquired in the time before the respective exercise session.

In an example, the exercise data comprise at least one of: a date and/or time of an exercise session comprising at least one exercise, at least one activity, and a corresponding duration, distance, number of repetitions, intensity, and/or speed.

This may help the user to correctly execute and schedule the suggested exercises.

The data and/or time may be a suggestion when to best perform the respective exercise (session). The time and/or data suggestions may be adjusted to one another such that, e.g., sufficient breaks may be scheduled between subsequent exercise (sessions). The time may also refer to how long to perform the respective exercise.

The activity may relate, e.g., to an exercise of the ongoing and/or upcoming exercise session or a full list of exercises and/or exercise sessions and may refer broadly to an activity like stretching, cycling, walking etc. and/or to specific exercises as a part thereof, e.g., in the example of stretching specific stretching exercises. With the activity, instructions may for example be provided, e.g., explaining how to exactly perform the respective exercise, e.g., by listing multiple steps of the respective exercise, errors to avoid, etc.

A number of sets and/or repetitions, duration, distance, and/or a calorie goal may be assigned to each of the exercises of the respective exercise session in terms of the associated intensity.

Further, the setting and/or specification of at least on exercise device may be included, e.g., when the user is using an exercise device in the respective exercise session. For example, when weights or exercise machines are used, their load and/or exact settings may, for example, be provided and adjusted in a small loop during the exercise.

A speed at which to perform a respective exercise/activity may be provided, e.g., for activities like walking, jogging, cycling, etc.

In another example, in a small loop, the at least one new and/or updated exercise parameter may be determined during an ongoing exercise for live adjustment of the ongoing exercise and/or, in a large loop, the at least one new and/or updated exercise parameter may be determined after a completed exercise for adjustment of an upcoming exercise.

This may provide a well-adjusted set of exercise parameters during the respective ongoing and/or upcoming exercise sessions.

The small loop may for example occur on a time scale of seconds to minutes, essentially time scales comparable to the duration of parts of individual exercises or up to the duration of the exercise session. In such small loop, the apparatus may correct, e.g., the intensity or execution of an exercise during the exercise itself and may thus provide quasi-instantaneous feedback.

The large loop may for example occur on a time scale of hours to days or weeks, essentially time scales comparable to the duration spanned by multiple consecutively scheduled exercise sessions.

An exemplary apparatus may update the respective exercise parameters in a small and in a large loop simultaneously, alternatingly, and/or alternatively to each other.

In another example, the apparatus may further comprise a user interface for outputting the dynamical patient data and/or the new and/or updated exercise parameter; and/or it may further be configured to send the dynamical patient data and/or the new and/or updated exercise parameter to a user device comprising a user interface for outputting the dynamical patient data and/or the new and/or updated exercise parameter.

This may improve user satisfaction and help the user to execute the suggested exercises correctly and effectively.

In an example, the user interface may also output static patient data and/or external data. Analogously, the apparatus may for example further be configured to send the static patient data and/or external data to a user device comprising a user interface for outputting.

The user interface may provide, e.g., visual, audible, and/or haptic feedback to the user. E.g., visual feedback may be provided via a screen. Alternatively or additionally, audible feedback may comprise spoken instructions and/or instructive sounds, e.g., a warning sound indicating the start, end, and/or correct/wrong execution of the exercise. Alternatively or additionally, haptic feedback may for example be provided via vibrating means and indicate, e.g., the start, end, and/or correct/wrong execution of the exercise.

In an exemplary embodiment, the apparatus may determine the new and/or updated exercise parameters such as to keep the at least one physiological parameter within a predetermined range during an ongoing and/or an upcoming exercise.

This may improve safety and efficiency of the respective exercise (session).

The predetermined range may be defined by a predetermined lower limit and a predetermined upper limit. Keeping the at least one physiological parameter within a predetermined range may, e.g., in the small loop, relate to adjusting the exercise parameters during an ongoing exercise. E.g., when the heart rate becomes too high, the suggested running speed, distance, and/or duration may be reduced and vice versa. Further, in the large loop, suggestions relating to upcoming exercises may be adjusted accordingly.

The apparatus may, for example be further configured to send an alarm signal to the patient when at least one critical physiological parameter leaves the predetermined range during an ongoing exercise, e.g., instructing the patient to reduce the intensity of the exercise or to stop it.

In an example, the apparatus may compare its expected heart rate for the given current exercise program with the actual measured heart rate and measured exercise. Based on these deviations the apparatus may adjust its expectations what extent of exercise will induce which increase in heart rate. Based on the adjusted expectations an adapted current and/or future exercise program can be suggested.

In an example, the apparatus may further be configured to provide the dynamical patient data and/or the new and/or updated exercise parameter(s) to a remote device.

The remote device may, e.g., comprise a database for storing the dynamical patient data, the static patient data, and/or the external data in a database. This may improve the reliability of the suggestions provided by the system and improve treatment/recovery success chances.

Storing the dynamical patient data, static patient data, current, new, and/or updated exercise data, and/or external data in such database may provide a patient log which may be stored on the apparatus, in an online database, and/or in an electronic patient data file accessible by authorized health professionals. Storing such data may provide the possibility to determine long-term developments and patterns to improve the performance of the apparatus, especially in suggesting the updated and/or new exercise parameters.

In another example, the means for determining may be configured to determine the at least one new and/or updated exercise parameter at least in part based on feedback from the remote device.

For example, this may be implemented in an exemplary embodiment in which the remote device may store, e.g., the dynamical patient data, static patient data, current, new, and/or updated exercise data, and/or external data in a database and provide at least some of those data, a processed version thereof, and/or further feedback based at least in part on those data to the apparatus in their original form. The remote device may, e.g., be configured to process the dynamical patient data, static patient data, current, new, and/or updated exercise data, and/or external data or at least a part thereof for determining a suitable new and/or updated exercise parameter and may provide that suggestion as feedback to the apparatus. The remote device may determine the new and/or updated exercise parameter by computational means, e.g., artificial intelligence-based means.

In an example, the apparatus may further comprise an artificial intelligence-based assistant for determining the at least one new and/or updated exercise parameter based at least in part on the dynamical patient data and the exercise data. Alternatively, the artificial intelligence-based assistant may be arranged in the remote device.

The artificial intelligence-based assistant may base its suggestions on all data stored in a database, e.g., as described herein. The database may for example be an EHR comprising electronic patient files. This may improve the over-all adjustment of the exercise plan to the patient's needs considering long-term trends apparent from the data collected in the database.

The artificial intelligence-based assistant may have access to dynamical and static patient data, exercise data, and/or external data from the respective patient as well as other patients which may be used for training of the artificial intelligence-based assistant.

The artificial intelligence-based assistant may be based on expert system, machine learning, deep learning, recurrent neural networks or a combination of these techniques. The data driven solutions may be trained on data sets from patients where experts have graded the exercise program as favorable or non-favorable, on data sets representing clinical guidelines and/or on common recommendations. The artificial intelligence-based assistant may in addition add rule-based boundary conditions to ensure safety and stability of its recommendations. In a further implementation, the artificial intelligence-based assistant is fed back with the physiological parameter recorded during the patient's exercise according to a prior recommendation of the artificial intelligence-based assistant to learn adaptations for the individual patient.

According to another aspect of the invention, a system may be provided. The system comprises an apparatus as described herein, and a sensor configured to acquire the dynamical patient data comprising the physiological parameter and to provide the dynamical patient data to the apparatus. Preferably, the sensor is an implantable device.

The interplay of apparatus and one or more sensors may be beneficial in establishing a system supporting the exercising of a patient. For example, a closed-loop configuration may thus be provided, in which the result of the exercise according to the suggested at least one exercise parameter may be monitored directly via the sensor to improve the small and/or large loop operation of such system, as outlined above.

The sensor being an implant may be particularly suitable to consistently provide up-to-data, reliable and continuous physiological data.

Analogously, a system may be provided, wherein the system comprises an implant as described herein and a computer program comprising instructions which, when the computer program is executed, cause an apparatus to perform any of the functionalities described herein.

For communication between the (at least one) sensor and the apparatus different scenarios may be considered. The sensor might be a wearable device or preferably an implant. The apparatus may be a mobile device, a (remote) server, a cloud-based server, a wearable device, etc.

If the apparatus is incorporated into the sensor device, e.g. a wearable device, then the communication between the sensor and the apparatus may use the technical infrastructure of the wearable device-this is usually a bus system which connects the processor (CPU) and the sensor hardware, e.g. SPI, I²C.

If the apparatus is a mobile device (e.g. a smartphone) which is usually in near distance to the sensor, then a wireless communication technology applicable within a short range (a few meters) may be used, e.g. BLE (Bluetooth Low Energy), UWB (Ultra Wideband), Zigbee or MICS (Medical implant communication service).

If the apparatus is remotely located with respect to the sensor, i.e. the apparatus is only accessible via internet, then the sensor may use a wide distance communication technology. Either a standard telecommunication technology such as LTE, GSM or Edge, or a wireless local area network technology such as WiFi may be used. If the sensor only contains a non-rechargeable battery, then the latter (to some extent) power consuming technologies might not be applicable. Instead, a near distance communication intermediate which bridges the communication from the sensor to the internet may be used. This communication intermediate may provide low power short range communication technology such as BLE, UWB, Zigbee or MICS to connect the sensor.

In an example, the system may further comprise a user device configured to receive the dynamical patient data and/or the new and/or updated exercise parameter and to provide them to a user via a user interface. Alternatively, the apparatus of the system may comprise a user interface for providing the dynamical patient data and/or the new and/or updated exercise parameter.

The user device may for example be a mobile phone, a wearable device, and/or a sensor-specific device. Generally, any of the functionalities described herein in reference to the apparatus and/or the sensor may also be implemented fully or in part in such user device.

According to a further aspect of the invention, a computer program is provided, wherein the computer program comprises instructions which, when the computer program is executed, cause an apparatus to receive dynamical patient data comprising at least one physiological parameter, and to receive exercise data comprising a current exercise parameter, and to determine a new and/or updated exercise parameter of an ongoing and/or an upcoming patient exercise, based at least in part on the dynamical patient data and the exercise data.

Such computer program provides an advantageous way to automize providing exercise parameter(s) of an ongoing and/or an upcoming patient exercise tailored to the patient's needs. This may improve reliability and suitability of the as-provided exercise parameter(s) and improve safety of the patient during the exercise.

According to a further aspect of the invention, a method is provided, which comprises steps according to the instructions of the computer program as described herein.

Generally, any functionality described herein with reference to an apparatus may also be implemented as a step of a respective, e.g. computer-implemented, method and/or computer program and vice versa.

For example, a patient may be diagnosed with a heart disease, e.g. a myocardial infarct. The patient may be treated accordingly. After the diagnosis and/or the treatment, the patient may be connected to a system as outlined herein. An apparatus may be provided (e.g. a dedicated handheld device, a smartphone of the patient with a corresponding app, or a remote server), e.g. as described herein, for example for receiving the dynamical patient data from an implant of the patient. Correspondingly a further treatment and/or monitoring method of the patient may be carried out including exercises.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

FIG. 1 Schematic representation of a system comprising a sensor, an apparatus for providing exercise assistance and a remote database.

FIG. 2 Schematic representation of a workflow for providing new and/or updated exercise parameters in a large loop.

FIG. 3 Schematic representation of a workflow for providing new and/or updated exercise parameters in a small loop.

### DETAILED DESCRIPTION

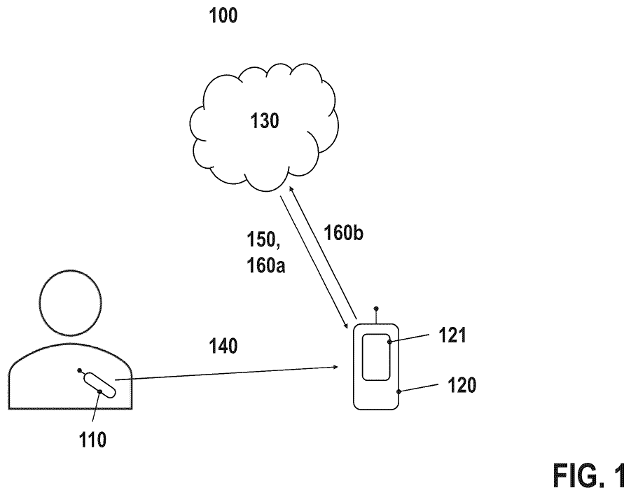

FIG. 1 shows a schematic representation of a system 100 comprising a sensor 110, an apparatus for providing exercise assistance 120 with a user interface 121 and a remote device 130 containing a data base, hereinafter also referred to as “database 130”. In the example of FIG. 1, the apparatus is implemented as a user device, e.g. a smartphone.

The sensor 110 of the exemplary embodiment of FIG. 1 is implanted into the patient's body and configured to acquire dynamical patient data 140 comprising at least one physiological parameter describing the health state of the patient. The sensor 110 transmits the dynamical patient data 140 to the apparatus 120 via wireless communication.

The apparatus 120 receives the dynamical patient data 140 and may process them in a data processing step along with optional additional static patient data 150, and exercise data 160a comprising parameters of previous and/or ongoing exercises that may be received from the database 130 and/or a memory of the apparatus 120, at each of which a log of static patient data 150 and exercise data 160a may be stored, from the user interface 121 of the apparatus 120, or from any other suitable source, e.g., a patient device like a mobile phone or a wearable device. Based on the dynamical patient data 140, the optional static patient data 150 and the exercise data 160a, the apparatus 120 provides exercise data 160b comprising new and/or updated exercise parameters. The apparatus provides the exercise data 160b to a user via the user interface 121 and/or transmits the exercise data 160b to the database 130 where they may be stored along with the exercise data 160a comprising exercise parameters of previous and/or ongoing exercises or to overwrite the exercise data 160a comprising parameters of previous and/or ongoing exercise(s).

In an exemplary workflow executed by the system 100 of FIG. 1, the implanted sensor 110 may record physiological data describing the health state of the patient and provide them, e.g., to the apparatus 120 directly or via for example a database 130, or a relay device. The apparatus 120 may communicate with a database 130, e.g., directly or via any of the devices described herein. The database 130 may store, e.g., dynamical patient data, static patient data, exercise data, and/or external data. The apparatus may receive the exercise data associated with a current exercise, e.g., from the database 130 and/or from any other source like, e.g., a user device or from a storage of the apparatus 120 itself, along with any data described herein that may be extracted from the database 130. All these data may be considered in a data processing step executed, e.g., in the apparatus 120. In such data processing step, new and/or updated exercise parameters may be determined. These may then be outputted via, e.g., a user interface 121 of the apparatus 120. The new and/or updated exercise parameters may further be added to the other data in the database 130 and stored, e.g., in form of a log file storing all previously determined exercise data. Generally, the interconnection between any of the members of the system 100 may be as described herein or optionally guided past other devices of the system 100 and/or additional (relay) devices.

It is noted that in other examples, the processing step may be performed, e.g. by a server which hosts database 130 and/or another server in communication with the apparatus 120.

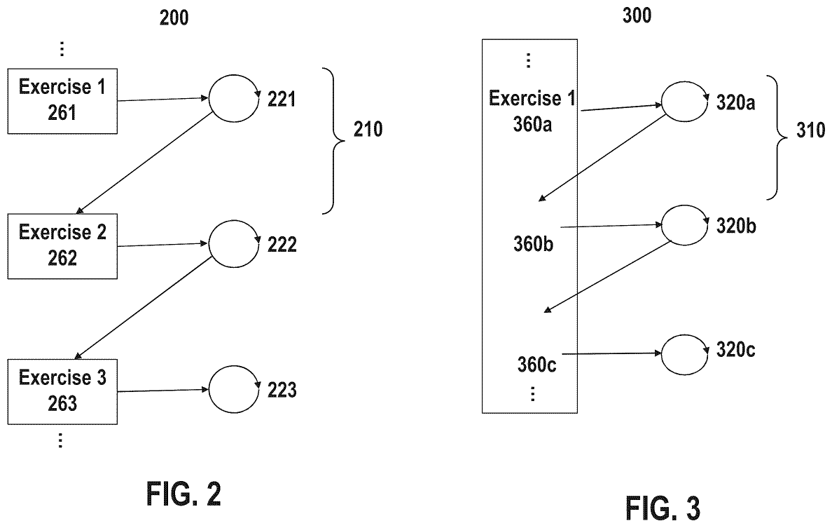

FIGS. 2 and 3 illustrate how the data processing step may be performed (repeatedly) in further detail.

FIG. 2 shows an exemplary schematic representation of a workflow 200 for providing new and/or updated exercise parameters in a large loop 210.

In the exemplary workflow 200 of FIG. 2, a patient executes an exercise 1 according to exercise data 261 comprising exercise parameters. In a subsequent and/or parallel data processing step 221, an apparatus (not shown) may provide new and/or updated exercise parameters to provide new and/or updated exercise data 262 based on the exercise data 261 and further data comprising, e.g., dynamical patient data pertaining to exercise 1 and static patient data (not shown). The new and/or updated exercise data 262 define an exercise 2 which may be performed at a later point in time compared to exercise 1. Hence, the processing step 221 may be part of a feedback loop, shown as large loop 210 in FIG. 1.

When the patient executes the second exercise (exercise 2) according to the new and/or updated exercise data 262, the exercise data 262 are the current exercise data. Based on, e.g., exercise data 262, and dynamical patient data pertaining to exercise 2, and optionally further data, again, new and/or updated exercise parameters forming new and/or updated exercise data 263 may be determined, e.g. in a processing step 222.

Exercise data 263 may then be used for a third exercise (exercise 3) which may occur at a later point in time compared to exercise 2. A further processing step 223 may be carried out such as to determine yet again new and/or updated exercise data, based on exercise data 263 and dynamical patient data pertaining to exercise 3. One or more further large loops may follow, as indicated by the three dots in FIG. 2.

Based on the loops, exercise data 261, 262, 263 may continuously be adjusted and thus also the as-developed exercise plans for upcoming exercise(s) according to the patient's needs by considering data from previous exercises.

It is noted that each processing step 221, 222, 223 may not only be based on the current or most recent exercise parameters and corresponding dynamical patient data but may generally use all available parameters and data pertaining to the patient.

FIG. 3 shows an exemplary schematic representation of a workflow 300 for providing new and/or updated exercise parameters in a small loop.

In the exemplary workflow 300 of FIG. 3, a patient executes a first exercise (exercise 1) throughout the workflow, in the beginning according to exercise data 360a comprising one or more exercise parameters. In a subsequent and/or parallel data processing step 320a, an apparatus (not shown) may provide new and/or updated exercise data 360b based on the exercise data 360a and further data comprising, e.g., dynamical and static patient data (not shown). This results in a modification of the ongoing exercise 1 according to the new and/or updated exercise data 360b. This is comprised in one small loop 310 which is repeated during the execution of the same first exercise according to new and/or updated exercise data 360b, 360c and a data processing steps 320b, 320c (and so forth). In this repetition, the new and/or updated exercise data 360c are determined based on the new and/or updated exercise data 360b in a data processing step 320b. The new and/or updated exercise data 360c may then be the basis for the further data processing step 320c. Updating exercise data 360a, 360b, 360c in a small loop 310 thus continuously adjusts the suggested exercise parameters for ongoing exercise(s) according to the patient's needs by considering data from previous exercises and the ongoing exercise.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. An apparatus for providing exercise assistance, comprising:
means for receiving dynamical patient data comprising at least one physiological parameter; means for receiving exercise data comprising a current exercise parameter; and
means for determining a new and/or updated exercise parameter of an ongoing and/or an upcoming patient exercise, based at least in part on the dynamical patient data and the exercise data.

2. The apparatus of claim 1, wherein the means for receiving the dynamical patient data are configured for receiving the dynamical patient data from an implantable device.

3. The apparatus of claim 1, wherein the dynamical patient data comprise at least one of a cardiovascular parameter, a respiratory parameter, and a motion-related parameter.

4. The apparatus of claim 1, wherein the apparatus further comprises means for receiving static patient data and/or external data; and the means for determining are further configured for determining the at least one new and/or updated exercise parameter, based at least in part on the static patient data and/or the external data.

5. The apparatus of claim 1, further comprising means for determining a new and/or updated exercise plan for an ongoing and/or an upcoming patient exercise, based at least in part on the at least one new and/or updated exercise parameter.

6. The apparatus of claim 1, wherein the exercise data comprise at least one of a date and/or time of an exercise session comprising at least one exercise, at least one activity, and a corresponding duration, distance, number of repetitions, intensity, and/or speed.

7. The apparatus of claim 1, wherein the at least one new and/or updated exercise parameter is determined during an ongoing exercise for live adjustment of the ongoing exercise; and/or the at least one new and/or updated exercise parameter is determined after a completed exercise for adjustment of an upcoming exercise.

8. The apparatus of claim 1, further comprising a user interface for outputting the dynamical patient data and/or the new and/or updated exercise parameter; and/or further configured to send the dynamical patient data and/or the new and/or updated exercise parameter to a user device comprising a user interface for outputting the dynamical patient data and/or the new and/or updated exercise parameter.

9. The apparatus of claim 1, wherein the new and/or updated exercise parameter is determined such as to keep the at least one physiological parameter within a predetermined interval during an ongoing and/or upcoming exercise.

10. The apparatus of claim 1, further configured to provide the dynamical patient data and/or the new and/or updated exercise parameter to a remote device.

11. The apparatus of claim 10, wherein the means for determining are configured to determine the at least one new and/or updated exercise parameter at least in part based on feedback from the remote device.

12. The apparatus of claim 1, further comprising an artificial intelligence-based assistant for determining the at least one new and/or updated exercise parameter based at least in part on the dynamical patient data and the exercise data.

13. A system comprising the apparatus of a claim 1; and a sensor configured to acquire the dynamical patient data comprising at least one physiological parameter and to provide the dynamical patient data to the apparatus; wherein preferably the sensor is an implantable device.

14. The system of claim 13, further comprising a user device configured to receive the dynamical patient data and/or the new and/or updated exercise parameter and to provide them to a user via a user interface; or wherein the apparatus comprises a user interface for providing the dynamical patient data and/or the new and/or updated exercise parameter.

15. A computer program comprising instructions which, when the computer program is executed, cause an apparatus to:
receive dynamical patient data comprising at least one physiological parameter;
receive exercise data comprising a current exercise parameter; and
determine a new and/or updated exercise parameter of an ongoing and/or an upcoming patient exercise, based at least in part on the dynamical patient data and the exercise data.
